SEQUENCE LISTING (42 sequences)

SEQ ID NO: 1 - protein, 56 aa, Streptococcus sp.
MEYKLILNGKTLKGETTTEAVDAATAEKVFKQYANDNGVDGEWTYDDATKTFTVTE

SEQ ID NO: 2 - DNA, 168 bp, Streptococcus sp.
  source 1..168: mol_type = genomic DNA
atggaatacaaactgatcctgaacggtaaaaccttaaaaggtgaaaccaccaccgaagcggttgatgcggcgaccgcggaaaaagttttcaaacagtatgccaacgataacggtgtggatggtgaatggacctacgatgatgctaccaaaaccttcactgttaccgaa

SEQ ID NO: 3 - protein, 238 aa, synthetic construct
  REGION 1..238: note = GFP
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDGPVLLPDNHYLSTQSALSKDPNEKRDHMVLLEFVTAAGITHGMDELYK

SEQ ID NO: 4 - DNA, 714 bp, synthetic construct
  misc_feature 1..714: note = GFP
  source 1..714: mol_type = other DNA
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaa

SEQ ID NO: 5 - protein, 34 aa, synthetic construct
  REGION 1..34: note = BiP
MARSFGANSTVVLAIIFFGCLFALSSAIEEATKL

SEQ ID NO: 6 - DNA, 272 bp, synthetic construct
  misc_feature 1..272: note = BiP
  source 1..272: mol_type = other DNA
atggctcgctcgtttggagctaacagtaccgttgtgttggcgatcatcttcttcggtgagtgattttccgatcttcttctccgatttagatctcctctacattgttgcttaatctcagaaccttttttcgttgttcctggatctgaatgtgtttgtttgcaatttcacgatcttaaaaggttagatctcgattggtattgacgattggaatctttacgatttcaggatgtttatttgcgttgtcctctgcaatagaagaggctacgaagtta

SEQ ID NO: 7 - DNA, 21 bp, synthetic construct
  misc_feature 1..21: note = 5' UTR
  source 1..21: mol_type = other DNA
ggcgtgtgtgtgtgttaaaga

SEQ ID NO: 8 - protein, 5 aa, synthetic construct
  REGION 1..5: note = EK
DDDDK

SEQ ID NO: 9 - DNA, 15 bp, synthetic construct
  misc_feature 1..15: note = EK
  source 1..15: mol_type = other DNA
gatgatgatgataag

SEQ ID NO: 10 - protein, 6 aa, synthetic construct
  REGION 1..6: note = TEV
ENLYFQ

SEQ ID NO: 11 - DNA, 18 bp, synthetic construct
  misc_feature 1..18: note = TEV
  source 1..18: mol_type = other DNA
gaaaacctgtacttccag

SEQ ID NO: 12 - DNA, 1220 bp, synthetic construct
  misc_feature 1..1220: note = MacT promoter
  source 1..1220: mol_type = other DNA
agagatctcctttgccccagagatcacaatggacgacttcctctatctctacgatctagtcaggaagttcgacggagaaggtgacgataccatgttcaccactgataatgagaagattagccttttcaatttcagaaagaatgctaacccacagatggttagagaggcttacgcagcaggtctcatcaagacgatctacccgagcaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgacgcaagacgtgacgtaagtatctgagctagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgt

SEQ ID NO: 13 - DNA, 520 bp, synthetic construct
  misc_feature 1..520: note = RD29B terminator
  source 1..520: mol_type = other DNA
aattttactcaaaatgttttggttgctatggtagggactatggggttttcggattccggtggaagtgagtggggaggcagtggcggaggtaagggagttcaagattctggaactgaagatttggggttttgcttttgaatgtttgcgtttttgtatgatgcctctgtttgtgaactttgatgtattttatctttgtgtgaaaaagagattgggttaataaaatatttgcttttttggataagaaactcttttagcggcccattaataaaggttacaaatgcaaaatcatgttagcgtcagatatttaattattcgaagatgattgtgatagatttaaaattatcctagtcaaaaagaaagagtaggttgagcagaaacagtgacatctgttgtttgtaccatacaaattagtttagattattggttaacatgttaaatggctatgcatgtgacatttagaccttatcggaattaatttgtagaattattaattaagatgttgattagttcaaacaaaaat

SEQ ID NO: 14 - protein, 60 aa, synthetic construct
  REGION 1..60: note = MP
ANITVDYLYNKETKLFTAKLNVNENVECGNNTCTNNEVHNLTECKNASVSISHNSCTAPD

SEQ ID NO: 15 - DNA, 180 bp, synthetic construct
  misc_feature 1..180: note = MP
  source 1..180: mol_type = other DNA
gcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgat

SEQ ID NO: 16 - protein, 158 aa, synthetic construct
  REGION 1..158: note = CBM3
VSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEP

SEQ ID NO: 17 - DNA, 471 bp, synthetic construct
  misc_feature 1..471: note = CBM3
  source 1..471: mol_type = other DNA
tctggtaacttgaaggttgaattttacaactctaacccatctgatactactaactctattaacccacaatttaaggttactaacactggttcttctgctattgatttgtctaagttgactttgagatactactacactgttgatggtcaaaaggatcaaactttttggtgtgatcatgctgctattattggttctaacggttcttacaacggtattacttctaacgttaagggtacttttgttaagatgtcttcttctactaacaacgctgatacttacttggaaatttcttttactggtggtactttggaaccaggtgctcatgttcaaattcaaggtagatttgctaagaacgattggtctaactacactcaatctaacgattactcttttaagtctgcttctcaatttgttgaatgggatcaagttactgcttacttgaacggtgttttggtttggggtaaggaacca

SEQ ID NO: 18 - protein, 77 aa, synthetic construct
  REGION 1..77: note = bdSUMO
HINLKVKGQDGNEVFFRIKRSTQLKKLMNAYCDRQSVDMTAIAFLFDGRRLRAEQTPDELEMEDGDEIDAMLHQTGG

SEQ ID NO: 19 - DNA, 231 bp, synthetic construct
  misc_feature 1..231: note = bdSUMO
  source 1..231: mol_type = other DNA
cacatcaacctcaaggtcaagggtcaggacggcaatgaggtgttcttccgcattaagaggtctacccagctgaagaagctgatgaatgcctactgcgaccgccagtctgtggacatgactgccattgccttcctgtttgatggtcgcaggctccgtgcagagcagactcctgacgagctcgagatggaagatggcgatgagatcgacgccatgcttcaccagactggaggc

SEQ ID NO: 20 - protein, 101 aa, synthetic construct
  REGION 1..101: note = LysM
GNTNSGGSTTTITNNNSGTNSSSTTYTVKSGDTLWGISQRYGISVAQIQSANNLKSTIIYIGQKLVLTGSASSTNSGGSNNSASTTPTTSVTPAKPTSQTT

SEQ ID NO: 21 - DNA, 303 bp, synthetic construct
  misc_feature 1..303: note = LysM
  source 1..303: mol_type = other DNA
ggtaatactaactctgggggttcaacgaccaccattacaaacaacaacagtggaacaaattcatcttcaaccacctacaccgtgaagagtggcgatacgttgtggggaatcagtcaacgttatggtattagcgttgctcagatccagtctgcaaataaccttaagtctactataatttatattgggcaaaagctagttctgactggctcggctagtagcaccaattccggaggtagcaataactcagcttctactacccctacaacctctgtaactccagctaagcctacatcacagactaca

SEQ ID NO: 22 - protein, 32 aa, synthetic construct
  REGION 1..32: note = mCor1
VSRLEEDVRNLNAIVQKLQERLDRLEETVQAK

SEQ ID NO: 23 - DNA, 96 bp, synthetic construct
  misc_feature 1..96: note = mCor1
  source 1..96: mol_type = other DNA
gtgtctaggcttgaggaagatgttagaaatctcaacgcaattgtccagaaacttcaggaaaggttggataggctggaggaaactgttcaagctaag

SEQ ID NO: 24 - DNA, 12 bp, synthetic construct
  misc_feature 1..12: note = HDEL
  source 1..12: mol_type = other DNA
cacgatgagctc

SEQ ID NO: 25 - protein, 184 aa, Homo sapiens
MVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 26 - DNA, 552 bp, Homo sapiens
  source 1..552: mol_type = genomic DNA
atggtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatg

SEQ ID NO: 27 - protein, 506 aa, Influenza A virus
DKICIGYQSTNSTETVDTLVENNVPVTHTKELLHTEHNGMLCATNLGHPLILDTCTIEGLVYGNPSCDLLLGGKEWSYIVERSSAVNGMCYPGRVENLEELRSFFSSARSYKRLLLFPDRTWNVTFNGTSKACSGSFYRSMRWLTHKNNSYPIQDAQYTNDWGKNILFMWGIHHPPTDTEQMNLYKKADTTTSITTEDINRTFKPGIGPRPLVNGQQGRIDYYWSVLKPGQTLRIRSNGNLIAPWYGHILSGESHGRILKTDLNSGNCIIQCQTEKGGLNTTLPFQNVSKYAFGNCPKYVGVKSLKLAVGLRNVPATSGRGLFGAIAGFIEGGWPGLVAGWYGFQHSNDQGVGIAADKESTQEAVDKITSKVNNIIDKMNKQYEIIDHEFSEIEARLNMINNKIDDQIQDIWAYNAELLVLLENQKTLDDHDANVNNLYNKVKRALGSNAIEDGKGCFELYHKCDDQCMETIRNGTYDRLKYKEESKLERQKIEGVKLESEETYKI

SEQ ID NO: 28 - DNA, 1518 bp, Influenza A virus
  source 1..1518: mol_type = genomic DNA
gataaaatttgcattggctaccagtcaacaaactccacagaaactgttgatacactagtagaaaacaatgtccctgtgacacataccaaagaattgctccacacagagcacaatggaatgttatgtgcaacaaacttgggacaccctcttatcctagacacctgcaccattgaagggttggtgtacggcaatccttcctgtgatttgctactgggagggaaagagtggtcttacattgtcgaaagatcatcagctgttaatgggatgtgctaccctggaagggtagagaatctggaagaactcaggtcctttttcagttctgctcgctcctacaaaagactcctactttttccagaccgtacttggaatgtgactttcaatgggacaagcaaagcatgctcaggctcattctacagaagtatgagatggctgacacacaagaacaattcttaccctattcaagacgcccaatataccaacgactggggaaagaatattctcttcatgtggggcatacaccatccacctactgatactgagcaaatgaatctatacaaaaaagctgatacaacaacaagtataacaacggaagatatcaatcgaactttcaaaccagggatagggccaaggcctcttgtcaatggtcaacaaggaagaattgattattattggtcagtactaaagccaggccagacattgcgaataagatccaatggaaatctaattgccccatggtatggacacattctttcaggagaaagccatggaagaatcctgaagaccgatttgaatagtggcaactgcataatacaatgccaaactgagaaaggtggtttgaacacgaccttgccattccaaaatgtcagcaaatatgcatttgggaactgtcccaaatatgttggagtgaagagtctcaaactggcagttggtctaaggaatgtgcctgctacatcaggtagagggcttttcggtgccatagctggattcatagaaggaggttggccaggactagttgcaggctggtacgggtttcagcactcaaatgatcaaggggttggaatagccgcagacaaagaatcaactcaagaagcagttgataaaataacatccaaagtaaataatataatcgacaaaatgaacaagcagtatgaaatcattgatcatgagttcagtgagattgaagccagactcaatatgatcaacaataagattgatgaccaaatacaggacatctgggcgtacaatgcagaattactagtactgcttgaaaaccagaaaacactcgatgatcatgatgcaaatgtgaacaatctgtataataaggtgaagagagcattgggttcaaatgcaatagaggatgggaagggatgcttcgagttgtatcacaaatgtgatgatcaatgcatggaaacaattagaaacgggacttatgacaggctaaagtataaagaagaatcaaaactagaaaggcagaaaatagaaggggtaaaactggagtctgaagaaacatacaagatt

SEQ ID NO: 29 - DNA, 35 bp, synthetic construct
  misc_feature 1..35: note = Primer
  source 1..35: mol_type = other DNA
gcggcgaccgcggcaaaagttttcaaacagtatgc

SEQ ID NO: 30 - DNA, 34 bp, synthetic construct
  misc_feature 1..34: note = Primer
  source 1..34: mol_type = other DNA
gcatactgtttgaaaacttttgccgcggtcgccg

SEQ ID NO: 31 - DNA, 25 bp, synthetic construct
  misc_feature 1..25: note = Primer
  source 1..25: mol_type = other DNA
gccttgcttcctattatatcttccc

SEQ ID NO: 32 - DNA, 29 bp, synthetic construct
  misc_feature 1..29: note = Primer
  source 1..29: mol_type = other DNA
cccggatccttgaacctcctgaacctccg

SEQ ID NO: 33 - DNA, 33 bp, synthetic construct
  misc_feature 1..33: note = Primer
  source 1..33: mol_type = other DNA
cggtgtggatggtgaagcgacctacgatgatgc

SEQ ID NO: 34 - DNA, 33 bp, synthetic construct
  misc_feature 1..33: note = Primer
  source 1..33: mol_type = other DNA
gcatcatcgtaggtcgcttcaccatccacaccg

SEQ ID NO: 35 - DNA, 25 bp, synthetic construct
  misc_feature 1..25: note = Primer
  source 1..25: mol_type = other DNA
gccttgcttcctattatatcttccc

SEQ ID NO: 36 - DNA, 29 bp, synthetic construct
  misc_feature 1..29: note = Primer
  source 1..29: mol_type = other DNA
cccggatccttgaacctcctgaacctccg

SEQ ID NO: 37 - DNA, 18 bp, synthetic construct
  misc_feature 1..18: note = Primer
  source 1..18: mol_type = other DNA
cagcagaacacccccatc

SEQ ID NO: 38 - DNA, 18 bp, synthetic construct
  misc_feature 1..18: note = Primer
  source 1..18: mol_type = other DNA
catgccgagagtgatccc

SEQ ID NO: 39 - DNA, 21 bp, synthetic construct
  misc_feature 1..21: note = Primer
  source 1..21: mol_type = other DNA
atggaaacattgtgctcagtg

SEQ ID NO: 40 - DNA, 19 bp, synthetic construct
  misc_feature 1..19: note = Primer
  source 1..19: mol_type = other DNA
ggtgctgagagaagccaag

SEQ ID NO: 41 - protein, 550 aa, Luciola cruciata
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 42 - DNA, 1653 bp, Luciola cruciata
  source 1..1653: mol_type = genomic DNA
caaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcaataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

(19) United States
(12) Patent Application Publication
(10) Pub. No.: US 2025/0011376 A1
(43) Pub. Date: Jan. 9, 2025

(54) EFFECT OF GB1 DOMAIN FUSION ON UPREGULATION OF RECOMBINANT PROTEIN EXPRESSION IN PLANT

(71) Applicant: POSTECH Research and Business Development Foundation, Pohang-si, Gyeongsangbuk-do (KR)

(72) Inventors: In Hwan HWANG, Pohang-si (KR); A Reum YUN, Pohang-si (KR); Byeong Ho MOON, Jeonju-si (KR); Shijian SONG, Pohang-si (KR); Haiping DIAO, Pohang-si (KR)

(73) Assignee: POSTECH Research and Business Development Foundation, Pohang-si, Gyeongsangbuk-do (KR)

(21) Appl. No.: 18/708,261

(22) PCT Filed: Sep. 15, 2022
(86) PCT No.: PCT/KR2022/013812

§ 371 Date: May 8, 2024

(30) Foreign Application Priority Data
Nov. 9, 2021 (KR) 10-2021-0153253

(51) Int. Cl. C07K 14/315; C07K 14/005; C07K 14/435; C07K 14/54; C12N 15/82

(52) U.S. Cl. CPC C07K 14/315; C07K 14/005; C07K 14/43595; C07K 14/5412; C12N 15/8242; C07K 2319/00; C12N 2760/16122

(57) ABSTRACT

The present invention relates to a GB1 domain fusion structure for upregulating recombinant protein expression in plants and, more specifically, to a GB1 domain fusion structure in which the GB1 domain of Streptococcus-derived protein G is fused with a target protein to be expressed in an upregulated manner, and a method for upregulating expression of a recombinant protein by using same. The present invention can bring about a reduced risk of contamination by pathogens and a high production yield, compared to the case of employing animal cells or microbes conventionally established to produce recombinant proteins, and a remarkable improvement in recombinant protein output, compared to the conventional case of employing plants. Thus, the present invention has several advantages in terms of quality and economy and thus can be a very competitive production method in the commercial production of recombinant proteins.

### TECHNICAL FIELD

The present invention relates to an effect of upregulating expression using GB1 domain fusion when expressing recombinant protein in plants and, more specifically, to a method of upregulating recombinant protein expression by fusing the GB1 domain of Streptococcus-derived protein G with a target protein to be expressed in plants to produce a GB1 domain fusion structure and using the same.

### BACKGROUND ART

In general, plants have great potential in the production of biopharmceutical proteins and peptides because they are easy to transform and economically inexpensive as protein materials. However, to date, most biopharmaceuticals have typically been produced by transforming cultured mammalian cells, bacteria, fungi, etc. However, compared to the mammalian cells, bacteria, and fungi, the production of therapeutic proteins in plants can exhibit a reduced risk of contamination by pathogens and a high production yield, and has several advantages in terms of quality as well as economy, as does production in seeds or other storage organs. In addition, plants can potentially produce recombinant proteins inexpensively, and the cultivation, harvesting, storage and processing of transgenic cereals can also use existing infrastructure and require only a relatively small capital investment, thereby making them a very competitive production method for the commercial production of recombinant proteins.

Therefore, the development of plant expression systems for transforming plant cells to produce useful proteins of interest is in the spotlight. However, one of the most major challenges in developing a platform for producing recombinant proteins using plants is the development of a system for mass production of useful proteins. As a method for increasing the expression level of recombinant proteins in plants, there have been proposed a method of improving recombinant protein expression by inserting a small domain with multiple N-glycosylation sites to induce glycosylation, and a method of utilizing highly efficient 5′-untranslated sequences (Kang et al. 2018, Sci. Rep. 8:4612; Kim et al. 2014, Nucleic Acids Res. 42: 485-498). However, the above method of improving recombinant protein expression in plants is successful in improving the protein level in plants to a certain level, but has the problem that the yield varies greatly depending on the target protein.

On the other hand, a difference in the production yield of recombinant proteins may be due to the unique properties of the target protein, and there may be a variety of different reasons depending on the target gene. However, when optimizing the codon of a heterologous gene for the expression host, the expression level of the target protein can be improved. In addition, the production of recombinant proteins can be upregulated by fusing water-soluble domains to the recombinant proteins. In fact, it has been reported that in E. coli, the solubility of the target protein can be increased by fusing the GST, MBP, and SUMO domains, thereby upregulating the production of recombinant proteins.

The present inventors have sought an effective method of improving the expression level when a recombinant protein is to be produced in a plant, and while doing so, have confirmed that when the GB1 domain derived from Streptococcus protein G is fused to the N-terminus of the target protein, the productivity of the recombinant protein in plants is significantly improved, thereby completing the present invention.

### DISCLOSURE

### Technical Problem

An object of the present invention is to provide a novel expression system for enhancing the amount of recombinant protein expression in plants.

### Technical Solution

In order to achieve the above object, the present invention provides a fusion protein including: a target protein; and a GB1 domain bound to the N-terminus of the target protein.

In the present invention, the GB1 domain may be represented by the amino acid sequence of SEQ ID NO. 1.

In the present invention, a cleavage site may be further included between the target protein and the GB1 domain.

In the present invention, the fusion protein may further include an intracellular organelle targeting sequence.

The present invention also provides a DNA construct comprising a nucleotide sequence encoding the fusion protein.

In the present invention, the DNA construct may further include a 5′ UTR sequence at the 5′-terminal site of the nucleotide sequence encoding the fusion protein.

In the present invention, the GB1 domain may be fused to the N-terminus of the target protein to increase the expression amount of the target protein in plants.

The present invention also provides a plant cell into which the DNA construct or a recombinant vector including the DNA construct is introduced.

In the present invention, the plant cell may be derived from a plant selected from a group consisting of Arabidopsis thaliana, soybean, tobacco, eggplant, red pepper, potato, tomato, Chinese cabbage, radish, cabbage, lettuce, peach, pear, strawberry, watermelon, melon, cucumber, carrot, celery, rice, barley, wheat, rye, corn, sugarcane, oats, and onions.

The present invention also provides a method for producing a target protein in a plant cell, the method including the steps of.
(a) culturing the plant cell; and
(b) recovering the target protein by crushing the cultured plant cell.

In the present invention, when the DNA construct further includes a cleavage site between the target protein and the GB1 domain, the target protein and the GB1 domain may be cleaved to recover the target protein from which the GB1 domain has been removed.

The present invention also provides a transgenic plant into which the DNA construct or a recombinant vector including the DNA construct is introduced.

In the present invention, the transgenic plant may be selected from a group consisting of Arabidopsis thaliana, soybean, tobacco, eggplant, red pepper, potato, tomato, Chinese cabbage, radish, cabbage, lettuce, peach, pear, strawberry, watermelon, melon, cucumber, carrot, celery, rice, barley, wheat, rye, corn, sugarcane, oats, and onions.

The present invention also provides a method for producing a target protein in a transgenic plant, the method including the steps of:
(a) growing the transgenic plant; and
(b) recovering the target protein by crushing the tissue isolated from the plant.

In the present invention, when the DNA construct further includes a cleavage site between the target protein and the GB1 domain, the target protein and the GB1 domain may be cleaved to recover the target protein from which the GB1 domain has been removed.

### Advantageous Effects

The present invention can bring about a reduced risk of contamination by pathogens and a high production yield, compared to the case of employing animal cells or microbes conventionally established to produce recombinant proteins, and a remarkable improvement in recombinant protein output, compared to the conventional case of employing plants. Thus, the present invention has several advantages in terms of quality and economy and thus can be a very competitive production method in the commercial production of recombinant proteins.

### DESCRIPTION OF DRAWINGS

FIG. 1 shows 5′UTR::BiP:GFP:HDEL (control) and 5′UTR::BiP:GB1:TEV:EK:GFP:HDEL (GB1 fusion experiment group) targeted to endoplasmic reticulum, 5′UTR::RbcS:GFP:HDEL (control) and 5′UTR::RbcS:GB1:EK:GFP:HDEL (GB1 fusion experiment group) targeted to chloroplast, and 5′UTR:GFP:HDEL (control) and 5′UTR::GB1:GFP:HDEL (GB1 fusion experiment group) targeted to cytoplasm.

FIG. 2 shows the results of inducing the expression of GB1-fused GFP and GB1-unfused GFP in endoplasmic reticulum, chloroplast and cytoplasm, and comparing their expression levels. FIG. 2A shows the results of confirming the expression levels of GB1-fused GFP and GB1-unfused GFP, both targeting the endoplasmic reticulum, by fluorescence microscopy 5 and 7 days after Agrobacterium-mediated transformation, respectively; and FIG. 2B shows the results of confirming the expression levels of GB1-fused GFP and GB1-unfused GFP, both targeting the endoplasmic reticulum, by SDS-PAGE and Coomassie brilliant blue staining 3, 5, and 7 days after Agrobacterium-mediated transformation, respectively. FIG. 2C shows the results of confirming the expression levels of GB1-fused GFP and GB1-unfused GFP, both targeting the chloroplast, by fluorescence microscopy 5 and 7 days after Agrobacterium-mediated transformation, respectively; and FIG. 2D shows the results of confirming the expression levels of GB1-fused GFP and GB1-unfused GFP, both targeting the chloroplast, by SDS-PAGE and Coomassie brilliant blue staining 3, 5, and 7 days after Agrobacterium-mediated transformation, respectively. FIG. 2E shows the results of confirming the expression levels of GB1-fused GFP and GB1-unfused GFP, both targeting the cytoplasm, by fluorescence microscopy 5 and 7 days after Agrobacterium-mediated transformation, respectively; and FIG. 2F shows the results of confirming the expression levels of GB1-fused GFP and GB1-unfused GFP, both targeting the cytoplasm, by SDS-PAGE and Coomassie brilliant blue staining 3, 5, and 7 days after Agrobacterium-mediated transformation, respectively.

FIG. 3 shows the results of comparing the GFP expression amounts in each of GFP without GB1 fused, GFP with GB1 fused to the N-terminus, and GFP with GB1 fused to the C-terminus. FIG. 3A shows a schematic diagram of DNA constructs for comparing the expression of GFP without GB1 fused, GFP with GB1 fused to the N-terminus, and GFP with GB1 fused to the C-terminus, all targeting the endoplasmic reticulum; FIG. 3B shows the results of confirming the GFP expression amounts by fluorescence microscopy 3, 5 and 7 days after Agrobacterium-mediated transformation with these constructs; FIG. 3C shows the results of confirming the GFP expression amounts by immunoblotting 3, 5 and 7 days after Agrobacterium-mediated transformation with these constructs; FIG. 3D shows the results of confirming the GFP expression amounts by Coomassie brilliant blue staining 3, 5 and 7 days after Agrobacterium-mediated transformation with these constructs; and FIG. 3E is the result of quantitatively representing FIG. 3D.

FIG. 4 shows the results of further verifying the effect of upregulating the expression of the target protein by fusing the GB1 domain to the HA of human IL6 and H9N2, respectively. FIG. 4A shows the constructs of human IL6 without GB1 fused, human IL6 with GB1 fused to the N-terminus, HA of H9N2 without GB1 fused, and HA of H9N2 with GB1 fused to the N-terminus. FIG. 4B shows the results of confirming the effect of upregulating the expression of human IL6 according to GB1 domain fusion through bead purification, and FIG. 4C is the result of quantifying the results of FIG. 4B. FIG. 4D shows the results of confirming the effect of upregulating the expression of HA of H9N2 according to GB1 domain fusion through bead purification, and FIG. 4E is the result of quantifying the results of FIG. 4D.

FIG. 5 shows the results of preparing a variant in which E27 is substituted with alanine or E27 and W43 are simultaneously substituted with alanine, and verifying whether the GB1 variant can exert the effect of upregulating the expression amount of the target protein in order to confirm the important amino acid sequence of the GB1 protein in the effect of enhancing the expression level of the target protein. FIG. 5A shows the amino acid sequences of the wild type and variant of GB1. GFP, GFP with a wild-type GB1 domain fused to the N-terminus, GFP with a variant GB1 (E27A) domain fused to the N-terminus, and GFP with a mutant GB1 (E27A & W43A) domain fused to the N-terminus were expressed in N. benthamiana, respectively, and the amount of GFP expression was observed with a fluorescence microscope (FIG. 5B) and quantified (FIG. 5C), and the Coomassie brilliant blue staining was performed (FIG. 5D) and quantified (FIG. 5E).

FIG. 6 shows the results of confirming the effect of upregulating the expression amount of the target protein by GB1 using Arabidopsis thaliana by quantitative RT-PCR.

FIG. 7 shows the results of confirming the effect of upregulating the expression amount of the target protein by GB1 at the translation stage using wheat germ extract in vitro.

### BEST MODES OF THE INVENTION

Unless otherwise defined, all technical and scientific terms used in this specification have the same meaning as commonly understood by a person skilled in the art to which the present invention pertains. In general, the nomenclature used in this specification and the experimental methods described below are well known and commonly used in the art.

In the present invention, it has been confirmed that when the GB1 domain of protein G derived from Streptococcus is fused to the target protein, the expression amount of the target protein in plants can be significantly improved. Specifically, in the present invention, cytoplasmic expression has been attempted to be induced by using green fluorescent protein (GFP) as a model protein and fusing GB1 to the N-terminus of GFP to produce a GB1-GFP construct. Since the endoplasmic reticulum and chloroplast in plant cells are the two main places for storing recombinant proteins, the leader sequence of BiP or the transit peptide of RbcS has been fused to the N-terminus of GB1-GFP to produce BiP-GB1-GFP or RbcS(tp)-GB1-GFP, and the expression has been induced in the endoplasmic reticulum and chloroplast, respectively.

As a result of transiently expressing the construct prepared as described above in Nicotiana benthamian, and comparing the expression amounts, it has been confirmed that the expression amount of GFP fused with GB1 is significantly improved in all of the cytoplasm, endoplasmic reticulum, and chloroplast.

On the other hand, it has been confirmed that when the GB1 domain is fused to the C-terminus of the target protein, the effect of improving the target protein is not exerted, and thus, it is especially important to fuse the GB1 domain to the N-terminus of the target protein. Further, it could be confirmed that E27 and W43, amino acids that bind to the antibody Fc region in the GB1 domain, are amino acid residues that play an important role in improving the expression level of the target protein by the GB1 domain.

In addition, it has been confirmed that the GB1 domain can effectively improve the expression level of human IL6 and HA of H9N2 as well as GFP, and can improve the expression amount of the target protein not only in Nicotiana benthamian but also in Arabidopsis thaliana.

In addition, it has been confirmed that the GB1 domain can improve the expression amount of the target protein at both the transcription and translation stages.

Therefore, in one aspect, the present invention relates to a fusion protein including: a target protein; and a GB1 domain bound to the N-terminus of the target protein.

In the present invention, the GB1 domain may be represented by the amino acid sequence of SEQ ID NO. 1, but is not limited thereto.

That is, not only the GB1 domain of protein G derived from Streptococcus, but also the GB1 domain of protein G derived from other organisms or microorganisms may be used to be fused with the target protein to improve the expression amount of the target protein in plants. In particular, in the present invention, the importance of the E27 and/or W43 residues of the GB1 domain represented by SEQ ID NO. 1 has been confirmed, and thus, it is preferable that the E27 residue or E27 and W43 are not mutated.

However, the amino acid residues or other residues of the GB1 domain may have a non-conserved substitution or a conserved substitution.

The amino acid substitution of the present invention may be a non-conserved substitution. The non-conserved substitution may include, for example, changing an amino acid residue of the target protein or polypeptide in a non-conservative manner, such as replacing an amino acid residue having a specific side chain size or specific property (e.g., hydrophilicity) with an amino acid residue having a different side chain size or different property (e.g., hydrophobicity).

The amino acid substitution may also be a conserved substitution. The conserved substitution may include, for example, changing an amino acid residue of the target protein or polypeptide in a conservative manner, such as replacing an amino acid residue having a specific side chain size or specific property (e.g., hydrophilicity) with an amino acid residue having the same or similar side chain size or the same or similar property (e.g., still hydrophilicity). Such a conserved substitution generally does not significantly affect the structure or function of the produced protein. In the present invention, an amino acid sequence variant that is a mutation of a fusion protein, a fragment thereof, or a variant thereof in which one or more amino acids are substituted may include a conserved amino acid substitution that does not significantly change the structure or function of the protein.

For example, a mutual substitution between amino acids in each of the following groups may be considered a conserved substitution in the present invention:

A group of amino acids with non-polar side chains: alanine, valine, leucine, isoleucine, proline, phenylalanine, tryptophan and methionine.

A group of uncharged amino acids with polar side chains: glycine, serine, threonine, cysteine, tyrosine, asparagine, and glutamine.

A group of negatively charged amino acids with polar side chains: aspartic acid and glutamic acid.

A group of positively charged basic amino acids: lysine, arginine, and histidine.

A group of amino acids with phenyl: phenylalanine, tryptophan and tyrosine.

The proteins, polypeptides and/or amino acid sequences encompassed by the present invention may also be understood to include at least the following range: variants or homologues having the same or similar function as the protein or polypeptide.

In the present invention, the variant may be a protein or polypeptide produced by substitution, deletion or addition of one or more amino acids compared to the amino acid sequence of the protein and/or the polypeptide. For example, the functional variant may include a protein or polypeptide with an amino acid change due to substitution, deletion and/or insertion of at least one amino acid, for example 1-30, 1-20 or 1-10 amino acids, alternatively 1, 2, 3, 4, or 5 amino acids. The functional variant may substantially retain the biological properties of the protein or polypeptide prior to the change (for example, substitution, deletion, or addition). For example, the functional variant may retain at least 60%, 70%, 80%, 90%, or 100% of the biological activity of the protein or polypeptide prior to the change.

In the present invention, the homologue may be a protein or polypeptide having at least about 80% (e.g., at least about 85%, about 90%, about 91%, about 92%, about 93%, about 94%, about 95%, about 96%, about 97%, about 98%, about 99% or more) sequence homology to the amino acid sequence of the protein and/or the polypeptide.

In the present invention, the homology generally refers to similarity, analogousness, or association between two or more sequences. “Percent of sequence homology” may be calculated by comparing two sequences arranged in a comparison window that determines the number of positions where the same nucleic acid base (e.g., Ala, Pro, Ser, Thr, Gly, Val, Leu, Ile, Phe, Tyr, Trp, Lys, Arg, His, Asp, Glu, Asn, Gln, Cys, and Met) is present, wherein the number of matching positions is divided by the total number of positions to provide the number of matching positions in the comparison window (i.e., window size), and the result is multiplied by 100 to give the percentage of sequence homology. The alignments for determining the percent of sequence homology may be performed in a variety of ways known in the art, for example, using publicly available computer software such as BLAST, BLAST-2, ALIGN or Megalign (DNASTAR) software. One skilled in the art may determine appropriate parameters for sequence alignment, including any algorithm required to achieve maximum alignment within full-length sequences or within the target sequence region to be compared. The homology can also be determined by the following methods: FASTA and BLAST. The FASTA algorithm is described, for example, in W. R. Pearson and D. J. Lipman's “Improved Tool for Biological Sequence Comparison”, Proc. Natl. Acad. Sci., 85: 2444-2448, 1988; and D, J. Lipman and W. R. Pearson's “Fast and Sensitive Protein Similarity Search”, Science, 227:1435-1441, 1989. For a description of the BLAST algorithm, see S. Altschul, W. Gish, W. Miller, E. W. Myers and D. Lipman, “A Basic Local Alignment Search Tool”, Journal of Molecular Biology, 215: 403-410, 1990.

In the present invention, the fusion protein may further include a cleavage site between the target protein and the GB1 domain. Such a cleavage site may be cleaved by a cleavage enzyme specific to the cleavage site in order to obtain the target protein by overexpressing the target protein in a plant and then separating only the target protein from the GB1 domain.

In the present invention, the cleavage site may be selected from the group consisting of an enterokinase cleavage site, a TEV cleavage site, a SUMO protease bdSENP cleavage site, a furin cleavage site, a thrombin cleavage site, and a 3C protease cleavage site, and a self-cleaving intein cleavage site, but is not limited thereto.

In the present invention, the cleavage enzyme may be selected from the group consisting of enterokinase, TEV, SUMO protease bdSENP, furin, thrombin, 3C protease, and self-cleaving intein, but is not limited thereto.

In the present invention, the fusion protein may further include an intracellular organelle targeting sequence.

The intracellular organelle may be selected from the group consisting of endoplasmic reticulum, chloroplast, vacuole (e.g., storage vacuole), apoplast, and cytoplasm, in which the target protein is produced within plant cells and stored or processed, but is not limited to thereto.

In the present invention, the sequence targeting the endoplasmic reticulum may preferably be BiP, amylase, or invertase sequence, but is not limited thereto.

In the present invention, the sequence targeting the storage vacuole may preferably be glutelin, globulin, prolamin, gluenin, phaseolin, or beta-conglycinin sequence, but is not limited thereto.

In the present invention, the sequence targeting the chloroplasts may preferably be RbcS, Cab, Tha4, rubisco activase, ferritin, or FtsH protease sequence, but is not limited thereto.

In another aspect, the present invention relates to a DNA construct comprising a nucleotide sequence encoding the fusion protein.

In the present invention, the DNA construct may further include a 5′ UTR sequence at the 5′-terminal site of the nucleotide sequence encoding the fusion protein.

In the present invention, the GB1 domain may be fused to the N-terminus of the target protein to increase the expression amount of the target protein in plants.

In still another aspect, the present invention relates to a plant cell into which the DNA construct or a recombinant vector including the DNA construct is introduced.

In the present invention, the recombinant vector may be a binary vector, a DNA viral vector, or an RNA viral vector, but is not limited thereto.

A preferred example of the recombinant vector of the present invention is a Ti-plasmid vector, which is capable of transferring a part of itself, the so-called T-region, to plant cells when present in a suitable host such as Agrobacterium tumefaciens. Another type of Ti-plasmid vector (see EP 0 116 718 B1) is currently used to transfer a hybrid DNA sequence to a plant cell, or a protoplast from which a new plant that appropriately inserts hybrid DNA into the plant's genome can be produced. A particularly preferred form of the Ti-plasmid vector is the so-called binary vecto as claimed in EP 0 120 516 B1 and U.S. Pat. No. 4,940,838. Other suitable vectors that may be used to introduce the DNA according to the invention into a plant host may be selected from viral vectors, for example, incomplete plant virus vectors, such as those that may be derived from double-stranded plant viruses (e.g., CaMV) and single-stranded viruses, geminiviruses, etc. The use of such vectors can be advantageous, particularly when it is difficult to properly transform a plant host.

The expression vector will preferably include one or more selective markers. The marker is a nucleic acid sequence having characteristics that can be generally selected by chemical methods, and includes all genes that can distinguish transformed cells from non-transformed cells. Examples thereof include herbicide resistance genes such as glyphosate or phosphinothricin, and antibiotic resistance genes such as Kanamycin, G418, Bleomycin, hygromycin, and chloramphenicol, but is not limited thereto.

In the plant expression vector of the present invention, the promoter may be CaMV 355, double enhancer CaMV, MacT, CsVMV, actin, ubiquitin, pEMU, MAS, or histone promoter, but is not limited thereto. The term “promoter” means a region upstream of DNA from a structural gene and refers to a DNA molecule to which RNA polymerase binds to initiate transcription. A “plant promoter” is a promoter capable of initiating transcription in plant cells. A “constitutive promoter” is a promoter that is active under most environmental conditions and developmental states or cell differentiation. The constitutive promoters may be preferred in the present invention because the selection of a transformant may be accomplished by various tissues at various stages. Therefore, the constitutive promoter does not limit the possibility of selection.

In the recombinant vector of the present invention, common terminators may be used, examples of which include nopaline synthase (NOS), rice α-amylase RAmy1 A terminator, HSP18.2 terminator, intro removal terminator of tobacco (Nicotiana tabacum) extensin, protease inhibitor II terminator, RD19B terminator, phaseoline terminator, terminator of Octopine gene of Agrobacterium tumefaciens, rmB1/B2 terminator of Escherichia coli, etc., but are not limited thereto. With respect to the need for terminators, it is generally known that such regions increase the certainty and efficiency of transcription in plant cells. Therefore, the use of terminators is highly preferred in the context of the present invention.

In the present invention, the plant cell may be a plant cell derived from a dicotyledon or a monocotyledon, but is not limited thereto.

In the present invention, the dicotyledon may be selected from the group consisting of soybean, tobacco, eggplant, red pepper, potato, tomato, Chinese cabbage, radish, cabbage, lettuce, peach, pear, strawberry, watermelon, melon, cucumber, carrot and celery, but is not limited thereto.

Additionally, in the present invention, the monocotyledon may be selected from the group consisting of rice, barley, wheat, rye, com, sugarcane, oats, and onions, but is not limited thereto.

In some aspect, the plant cell may be derived from Nicotiana benthamiana, Nicotiana tabacum or Arabidopsis thaliana.

In another aspect, the present invention relates to a method for producing a target protein in a plant cell, the method including the steps of
(a) culturing the plant cell; and
(b) recovering the target protein by crushing the cultured plant cell.

In the present invention, when the DNA construct further includes a cleavage site between the target protein and the GB1 domain, the target protein and the GB1 domain may be cleaved to recover the target protein from which the GB1 domain has been removed.

In still another aspect, the present invention relates to a transgenic plant into which the DNA construct or a recombinant vector including the DNA construct is introduced.

In the present invention, the transgenic plant may be a dicotyledon or a monocotyledon, but is not limited thereto.

In the present invention, the dicotyledon may be selected from the group consisting of soybean, tobacco, eggplant, red pepper, potato, tomato, Chinese cabbage, radish, cabbage, lettuce, peach, pear, strawberry, watermelon, melon, cucumber, carrot and celery, but is not limited thereto.

Additionally, in the present invention, the monocotyledon may be selected from the group consisting of rice, barley, wheat, rye, com, sugarcane, oats, and onions, but is not limited thereto.

In some aspect, the transgenic plant may be Nicotiana benthamiana, Nicotiana tabacum or Arabidopsis thaliana.

In another aspect, the present invention relates to a method for producing a target protein in a transgenic plant, the method including the steps of:
(a) growing the transgenic plant; and
(b) recovering the target protein by crushing the tissue isolated from the plant.

In the present invention, when the DNA construct further includes a cleavage site between the target protein and the GB1 domain, the target protein and the GB1 domain may be cleaved to recover the target protein from which the GB1 domain has been removed, but is not limited thereto.

As used herein, a “vector” refers to a DNA product containing a DNA sequence operably linked to a suitable regulatory sequence capable of expressing the DNA in a suitable host. The vector may be a plasmid, phage particle, or simply a potential genomic insert. Once transformed into a suitable host, the vector can replicate and function independently of the host genome, or in some cases can be integrated into the genome itself. Since the plasmid is currently the most commonly used form of vector, the “plasmid” and the “vector” are sometimes used interchangeably in the specification of the present invention. For the purposes of the present invention, it is preferred to use a plasmid vector. A typical plasmid vector that can be used for this purpose has a structure comprising (a) an origin of replication that allows efficient replication to contain several to hundreds of plasmid vectors per host cell, (b) an antibiotic resistance gene that allows selection of host cells transformed with the plasmid vector, and (c) a restriction enzyme cleavage site into which a foreign DNA fragment can be inserted. Even if an appropriate restriction enzyme cleavage site does not exist, the vector and foreign DNA can be easily ligated using a synthetic oligonucleotide adapter or linker according to a conventional method. After ligation, the vector must be transformed into an appropriate host cell. Transformation can be easily achieved using the calcium chloride method, electroporation (Neumann, et al., EMBO J., 1:841, 1982), or the like.

The vector used to overexpress the gene according to the present invention may be an expression vector known in the art. In the present invention, a binary vector commonly used for transformation of a plant was used.

As well known in the art, in order to increase the expression level of a transgenic gene in a host cell, the gene must be operably linked to transcriptional and decoding expression regulation sequences. Preferably, the expression regulation sequence and the corresponding gene are contained in one recombinant vector that containing a bacterial selection marker and a replication origin together. The recombinant vector preferably further contains an expression marker useful in plant cells. A plant or a plant cell transformed by the above-described recombinant vector constitute another aspect of the present invention. As used herein, the term “transformation” refers to introducing DNA into a host so that the DNA can be replicated as an extrachromosomal factor or by completion of chromosomal integration. Meanwhile, “transfection” refers to introducing DNA into a host cell so that it can be replicated within the host cell.

Of course, it should be understood that not all vectors are equally functional in expressing DNA sequences within the system of the present invention. However, those skilled in the art can make an appropriate selection among various vectors and expression control sequences without excessive experimental burden and without departing from the scope of the present invention. The copy number of the vector, the ability to control copy number, and the expression of other proteins encoded by the vector, such as antibiotic markers, should also be considered.

In this way, a gene encoding a target protein can be transiently expressed or stably transformed in a transformed plant or plant cell through a vector.

Not only can the gene encoding the target protein be transiently expressed in a transformed plant or plant cell, but also the gene encoding the target protein can be introduced into the genome of the transformed plant or plant cell and exist as a chromosomal factor, thereby being stably transformed. It will be obvious to those skilled in the art to which the present invention pertains that the same effect is obtained by inserting the gene targeting the target protein into the plant genome chromosome.

In the present invention, the introduction of a vector containing a gene encoding a target protein or the chromosome insertion of a gene encoding a target protein may be performed by adding an Agrobacterium containing the vector containing a gene encoding the target protein to a population of plant cells and co-culturing it.

In one embodiment, the co-culture may be performed under dark conditions. The co-culture is performed while agitating a culture of Agrobacterium containing a vector including a gene encoding the target protein with plant cells, and thereafter, may further include a stationary culture step.

In this way, the gene encoding the target protein can be transiently expressed or stably transformed in plant cells through a vector.

The stationary culture is a method of culturing without stirring the culture medium while the container is stationary, and herein, can be used interchangeably with deposition without agitation.

The stationary culture may be included in the form of a single or intermittent culture. When the single stationary culture is included, for example, plant cells and cultures of Agrobacterium may be co-cultured with agitation, stationarily cultured, and then cultured with agitation again. When the intermittent stationary culture is included, a culture form, in which plant cells and cultures of Agrobacterium are co-cultured with agitation, and after stationary culture, co-cultured with agitation again, may be repeated several to dozens of times.

Specifically, the culture may be performed by co-culturing the plant cells and the culture of Agrobacterium containing a vector containing a gene encoding the target protein while stirring for 1 minute to 48 hours, followed by stationary culturing for 1 minute to 96 hours, and then culturing with agitation again for 1 to 10 days. The OD₆₀₀ of Agrobacterium added for co-culture may be 0.00001 to 2.0.

If the OD₆₀₀ of Agrobacterium is too low, there is a problem that the transformation infection rate for temporary expression is low, and if it is too high, there is a problem that the survival rate of host cells drastically decreases. Therefore, it is desirable to co-cultivate by adding Agrobacterium having OD₆₀₀ in the above-defined range.

In this case, the Agrobacterium may be an Agrobacterium commonly used for plant transformation, and for example, Agrobacterium tumefaciens or Agrobacterium rhizogenes may be used.

The plant transformation refers to any method of transferring DNA to a plant. Such transformation methods do not necessarily require a period of regeneration and/or tissue culture. The transformation of plant species is now common for plant species including a monocotyledon as well as a dicotyledon. In principle, any transformation method can be used to introduce the hybrid DNA according to the invention into suitable progenitor cells. The method may be suitably selected from calcium/polyethylene glycol method for protoplasts, electroporation of protoplasts, microinjection into plant elements, (DNA or RNA-coated) particle impact method of various plant elements, infection by (incomplete) viruses in Agrobacterium tumefaciens-mediated gene transfer by plant infiltration or transformation of mature pollen or microspore, and the like. A preferred method according to the invention includes Agrobacterium-mediated DNA transfer.

In the present invention, the target protein may be, for example, any one or more target proteins selected from the group consisting of antigens, antibodies, antibody fragments, structural proteins, regulatory proteins, toxin proteins, hormones, hormone analogs, cytokines, enzymes, enzyme inhibitors, transport proteins, receptors, receptor fragments, biodefense inducers, storage proteins, movement proteins, exploitative proteins, and reporter proteins, but is not limited thereto.

In one example of the present invention, the expression was confirmed after transformation using Agrobacterium containing GFP (Green Fluorescent Protein), hIL6 or HA genes.

Such a high transformation expression rate indicates that the production of recombinant proteins at a commercial level is possible through transient expression.

The gene of the present invention may be subjected to various changes in the coding region as long as it does not change the amino acid sequence of the protein expressed from the coding region, and may be subjected to various changes or modifications even in parts other than the coding region to the extent that it does not affect the expression of the gene. Such modified genes are also included within the scope of the present invention.

Accordingly, the present invention also includes a polynucleotide having substantially the same base sequence as the gene, and a fragment of the gene. Substantially the same polynucleotide refers to a gene encoding an enzyme having the same function as that used in the present invention, regardless of sequence homology. The fragment of the gene also refers to a gene encoding an enzyme having the same function as that used in the present invention, regardless of the length of the fragment.

In addition, the protein, which is the expression product of the gene of the present invention, may be obtained from various biological resources such as microorganisms to the extent that its amino acid sequence does not affect the titer and activity of the enzyme, and the proteins obtained from such different biological resources are also included within the scope of the present invention.

Accordingly, the present invention also includes a polypeptide having substantially the same amino acid sequence as the protein, and a fragment of the polypeptide. Substantially the same polypeptide refers to a protein having the same function as that used in the present invention, regardless of amino acid sequence homology. The fragment of the polypeptide also refers to a protein having the same function as that used in the present invention, regardless of the length of the fragment.

### EXAMPLES

Hereinafter, the present invention will be described in more detail by way of examples. These examples are only for illustrating the present invention, and it will be obvious to those skilled in the art that the scope of the present invention is not interpreted as limited by these examples.

### Example 1. Preparation of GFP Expression Construct Targeting Endoplasmic Reticulum, Chloroplast and Cytoplasm

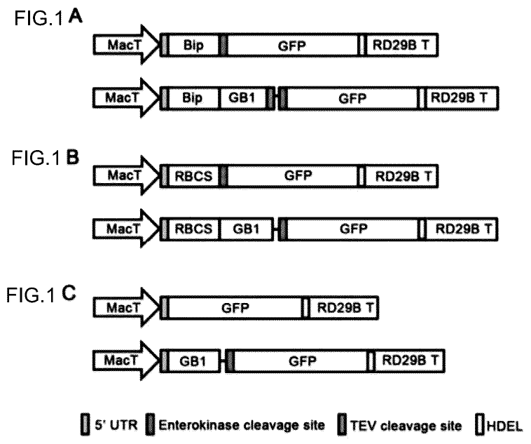

In order to confirm the effect of upregulating the expression of a protein fused with the GB1 domain, a total of six constructs were prepared: control constructs expressing GFP by targeting the endoplasmic reticulum, chloroplast, and cytoplasm, respectively; and experimental constructs expressing a fusion protein of GB1 and GFP by targeting the endoplasmic reticulum, chloroplast, and cytoplasm, respectively (see FIG. 1). Each of these constructs contained a 5′ UTR sequence at the N-terminal site and an HDEL sequence, an ER retention signal sequence, at the C-terminal site. Although the corresponding sequence is not required to reside in the chloroplast and cytoplasm, the corresponding sequence was also introduced into the constructs targeting the chloroplast and cytoplasm in order to ensure the same composition of the experimental protein. Meanwhile, an enterokinase cleavage site or a TEV cleavage site was included behind the BiP-GB1 or RbcS-GB1 domain so that it could be used when removing the GB1 domain from the GFP.

The amino acid sequence of the peptide or protein used in the present invention and the sequence encoding the same are described below, but this description is for illustrative purposes to specifically explain that the present invention can be operated. In this regard, it is obvious that a person skilled in the art can appropriately change and use the invention within an equivalent scope of the core technical idea in order to achieve the purpose of the present invention.

SEQ ID NO. 1: GB1 domain amino acid sequence
MEYKLILNGK TLKGETTTEA VDAATAEKVF KQYANDNGVD GEWTYDDATK TFTVTE
SEQ ID NO. 2: GB1 domain nucleotide sequence
atggaataca aactgatcct gaacggtaaa accttaaaag gtgaaaccac caccgaagcg 60
gttgatgcgg cgaccgcgga aaaagttttc aaacagtatg ccaacgataa cggtgtggat 120
ggtgaatgga cctacgatga tgctaccaaa accttcactg ttaccgaa
SEQ ID NO. 3: GFP amino acid sequence
MSKGEELFTG VVPILVELDG DVNGHKFSVS GEGEGDATYG KLTLKFICTT GKLPVPWPTL 60
VTTFSYGVQC FSRYPDHMKR HDFFKSAMPE GYVQERTIFF KDDGNYKTRA EVKFEGDTLV 120
NRIELKGIDF KEDGNILGHK LEYNYNSHNV YIMADKQKNG IKANFKTRHN IEDGGVQLAD 180
HYQQNTPIGD GPVLLPDNHY LSTQSALSKD PNEKRDHMVL LEFVTAAGIT HGMDELYK
SEQ ID NO. 4: GFP nucleotide sequence
atgagtaaag gagaagaact tttcactgga gttgtcccaa ttcttgttga attagatggt 60
gatgttaatg ggcacaaatt ttctgtcagt ggagagggtg aaggtgatgc aacatacgga 120
aaacttacce ttaaatttat ttgcactact ggaaaactac ctgttccatg gccaacactt 180
gtcactactt tctcttatgg tgttcaatgc ttttcaagat acccagatca tatgaagcgg 240
cacgacttct tcaagagcgc catgcctgag ggatacgtgc aggagaggac catcttcttc 300
aaggacgacg ggaactacaa gacacgtgct gaagtcaagt ttgagggaga caccctcgtc 360
aacaggatcg agcttaaggg aatcgatttc aaggaggacg gaaacatcct cggccacaag 420
ttggaataca actacaactc ccacaacgta tacatcatgg ccgacaagca aaagaacggc 480
atcaaagcca acttcaagac ccgccacaac atcgaagacg gcggcgtgca actcgctgat 540
cattatcaac aaaatactcc aattggegat ggccctgtcc ttttaccaga caaccattac 600
ctgtccacac aatctgccct ttcgaaagat cccaacgaaa agagagacca catggtcctt 660
cttgagtttg taacagctgc tgggattaca catggcatgg atgaactata caaa
SEQ ID NO. 5: BiP amino acid sequence
MARSFGANST VVLAIIFFGC LFALSSAIEE ATKL
SEQ ID NO. 6: BiP nucleotide sequence
atggctcgct cgtttggagc taacagtacc gttgtgttgg cgatcatctt cttcggtgag 60
tgattttccg atcttcttct ccgatttaga tctcctctac attgttgett aatctcagaa 120
ccttttttcg ttgttcctgg atctgaatgt gtttgtttgc aatttcacga tcttaaaagg 180
ttagatctcg attggtattg acgattggaa tctttacgat ttcaggatgt ttatttgcgt 240
tgtcctctgc aatagaagag gctacgaagt ta
SEQ ID NO. 7: 5′ UTR sequence
ggcgtgtgtgtgtgttaaaga
SEQ ID NO. 8: EK amino acid sequence
DDDDK
SEQ ID NO. 9: EK nucleotide sequence
gatgatgatgataag
SEQ ID NO. 10: TEV amino acid sequence
ENLYFQ
SEQ ID NO. 11: TEV nucleotide sequence
gaaaacctgtacttccag
SEQ ID NO. 12: MacT promoter sequence
agagatctcc tttgccccag agatcacaat ggacgacttc ctctatctct acgatctagt 60
caggaagttc gacggagaag gtgacgatac catgttcacc actgataatg agaagattag 120
ccttttcaat ttcagaaaga atgctaaccc acagatggtt agagaggctt acgcagcagg 180
tctcatcaag acgatctacc cgagcaataa tctccaggag atcaaatacc ttcccaagaa 240
ggttaaagat gcagtcaaaa gattcaggac taactgcatc aagaacacag agaaagatat 300
atttctcaag atcagaagta ctattccagt atggacgatt caaggcttgc ttcacaaacc 360
aaggcaagta atagagattg gagtctctaa aaaggtagtt cccactgaat caaaggccat 420
ggagtcaaag attcaaatag aggacctaac agaactcgcc gtaaagactg gcgaacagtt 480
catacagagt ctcttacgac tcaatgacaa gaagaaaatc ttcgtcaaca tggtggagca 540
cgacacgctt gtctactcca aaaatatcaa agatacagtc tcagaagacc aaagggcaat 600
tgagactttt caacaaaggg taatatccgg aaacctcctc ggattccatt gcccagctat 660
ctgtcacttt attgtgaaga tagtggaaaa ggaaggtggc tcctacaaat gccatcattg 720
cgataaagga aaggccatcg ttgaagatgc ctctgccgac agtggtccca aagatggacc 780
cccacccacg aggagcatcg tggaaaaaga agacgttcca accacgtctt caaagcaagt 840
ggattgatgt gacgcaagac gtgacgtaag tatctgagct agtttttatt tttctactaa 900
tttggtcgtt tatttcggcg tgtaggacat ggcaaccggg cctgaatttc gcgggtattc 960
tgtttctatt ccaacttttt cttgatccgc agccattaac gacttttgaa tagatacgct 1020
gacacgccaa gcctcgctag tcaaaagtgt accaaacaac gctttacagc aagaacggaa 1080
tgcgcgtgac gctcgcggtg acgccatttc gccttttcag aaatggataa atagccttgc 1140
ttcctattat atcttcccaa attaccaata cattacacta gcatctgaat ttcataacca 1200
atctcgatac accaaatcgt
SEQ ID NO. 13: RD29B terminator sequence
aattttactc aaaatgtttt ggttgctatg gtagggacta tggggttttc ggattccggt 60
ggaagtgagt ggggaggcag tggcggaggt aagggagttc aagattctgg aactgaagat 120
ttggggtttt gcttttgaat gtttgcgttt ttgtatgatg cctctgtttg tgaactttga 180
tgtattttat ctttgtgtga aaaagagatt gggttaataa aatatttgct tttttggata 240
agaaactctt ttagcggccc attaataaag gttacaaatg caaaatcatg ttagcgtcag 300
atatttaatt attcgaagat gattgtgata gatttaaaat tatcctagtc aaaaagaaag 360
agtaggttga gcagaaacag tgacatctgt tgtttgtacc atacaaatta gtttagatta 420
ttggttaaca tgttaaatgg ctatgcatgt gacatttaga ccttatcgga attaatttgt 480
agaattatta attaagatgt tgattagttc aaacaaaaat
SEQ ID NO. 14: MP amino acid sequence
ANITVDYLYN KETKLFTAKL NVNENVECGN NTCTNNEVHN LTECKNASVS ISHNSCTAPD
SEQ ID NO. 15: MP nucleotide sequence
gcaaacatca ctgtggatta cttatataac aaggaaacta aattatttac agcaaagcta 60
aatgttaatg agaatgtgga atgtggaaac aatacttgca caaacaatga ggtgcataac 120
cttacagaat gtaaaaatgc gtctgtttcc atatctcata attcatgtac tgctcctgat
SEQ ID NO. 16: CBM3 amino acid sequence
VSGNLKVEFY NSNPSDTTNS INPQFKVTNT GSSAIDLSKL TLRYYYTVDG QKDQTFWCDH 60
AAIIGSNGSY NGITSNVKGT FVKMSSSTNN ADTYLEISFT GGTLEPGAHV QIQGRFAKND 120
WSNYTQSNDY SFKSASQFVE WDQVTAYLNG VLVWGKEP
SEQ ID NO. 17: CBM3 nucleotide sequence
tctggtaact tgaaggttga attttacaac tctaacccat ctgatactac taactctatt 60
aacccacaat ttaaggttac taacactggt tcttctgcta ttgatttgtc taagttgact 120
ttgagatact actacactgt tgatggtcaa aaggatcaaa ctttttggtg tgatcatgct 180
gctattattg gttctaacgg ttcttacaac ggtattactt ctaacgttaa gggtactttt 240
gttaagatgt cttcttctac taacaacgct gatacttact tggaaatttc ttttactggt 300
ggtactttgg aaccaggtgc tcatgttcaa attcaaggta gatttgctaa gaacgattgg 360
tctaactaca ctcaatctaa cgattactct tttaagtctg cttctcaatt tgttgaatgg 420
gatcaagtta ctgcttactt gaacggtgtt ttggtttggg gtaaggaacc a
SEQ ID NO. 18: bdSUMO amino acid sequence
HINLKVKGQD GNEVFFRIKR STQLKKLMNA YCDRQSVDMT AIAFLFDGRR LRAEQTPDEL 60
EMEDGDEIDA MLHQTGG
SEQ ID NO. 19: bdSUMO nucleotide sequence
cacatcaacc tcaaggtcaa gggtcaggac ggcaatgagg tgttcttccg cattaagagg 60
tctacccagc tgaagaagct gatgaatgcc tactgcgacc gccagtctgt ggacatgact 120
gccattgcct tcctgtttga tggtcgcagg ctccgtgcag agcagactcc tgacgagctc 180
gagatggaag atggcgatga gatcgacgcc atgcttcacc agactggagg c
SEQ ID NO. 20: LysM amino acid sequence
GNTNSGGSTT TITNNNSGTN SSSTTYTVKS GDTLWGISQR YGISVAQIQS ANNLKSTIIY 60
IGQKLVLTGS ASSTNSGGSN NSASTTPTTS VTPAKPTSQT T
SEQ ID NO. 21: LysM nucleotide sequence
ggtaatacta actctggggg ttcaacgacc accattacaa acaacaacag tggaacaaat 60
tcatcttcaa ccacctacac cgtgaagagt ggcgatacgt tgtggggaat cagtcaacgt 120
tatggtatta gcgttgctca gatccagtct gcaaataacc ttaagtctac tataatttat 180
attgggcaaa agctagttct gactggctcg gctagtagca ccaattccgg aggtagcaat 240
aactcagctt ctactacccc tacaacctct gtaactccag ctaagcctac atcacagact 300
aca
SEQ ID NO. 22: mCorl amino acid sequence
VSRLEEDVRN LNAIVQKLQE RLDRLEETVQ AK
SEQ ID NO. 23: mCorl nucleotide sequence
gtgtctaggc ttgaggaaga tgttagaaat ctcaacgcaa ttgtccagaa acttcaggaa 60
aggttggata ggctggagga aactgttcaa gctaag
SEQ ID NO. 24: HDEL nucleotide sequence
cacgatgagc tc

### Example 2. Expression of GFP or GB1-GFP Fusion Protein in Endoplasmic Reticulum, Chloroplast, and Cytoplasm

The above six constructs targeting the endoplasmic reticulum, chloroplast, and cytoplasm were transformed into Agrobacterium (GV31010, EHA105, Intact Genomics, com.), and the transformed Agrobacterium was genetically introduced into Nicotiana benthamiana (Bio App Co., Ltd.) through syringe infiltration to induce transient expression.

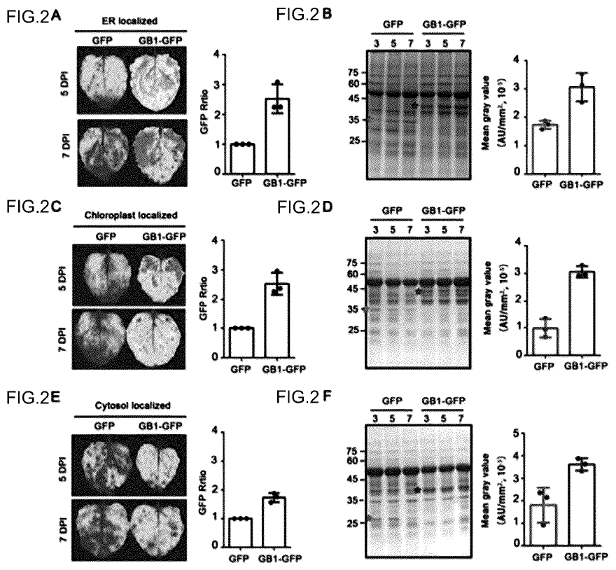

In order to confirm the GFP expression level, the fluorescence signals were activated at 488 nm after 5 and 7 days of transformation, respectively, and then the images were captured using a fluorescence microscope LAS3000. As a result, in all three locations within the cell, the endoplasmic reticulum, chloroplast, and cytoplasm, GFP with GB1 fusion showed a much higher fluorescence signal than GFP without GB1 fusion. As a result of calculating the fluorescence signal ratio to quantify the increase in GB1-mediated expression level, the fluorescence signal of GB1-fused GFP increased 2.5-fold, 2.5-fold, and 1.8-fold in the endoplasmic reticulum, chloroplast, and cytoplasm, respectively, compared to the case where GB1 was not fused, and thus, it was confirmed that the expression level of the recombinant protein could be significantly improved by fusion of the GB1 domain in all identified organelles (FIGS. 2A, 2C, and 2E).

Next, the effect of upregulating the expression level of the recombinant protein by fusion of the GB1 domain was verified by another method, Coomassie Brilliant Blue (CBB) staining. For this purpose, after 3, 5, and 7 days of transformation, total protein extract from N. benthamiana was quantified by Bradford assay, and 20 μg of total protein was developed by SDS-PAGE and then stained with Coomassie Brillent Blue (CBB) staining solution (CBB, 0.1%; methanol, 50%; glacial acetic acid, 10%) for 20 minutes. After staining, the stain was bleached with a washing solution containing 40% methanol and 10% glacial acetic acid, and the expression levels of the stained proteins were compared using the LAS3000 imaging system (Fuji, Japan).

The results also show a much higher level of GFP expression in all three locations of endoplasmic reticulum, chloroplast, and cytoplasm, compared to the case where the GFP was expressed alone (FIGS. 2B, 2D, 2E).

### Example 3. Comparison of Expression Amounts According to GB1 Domain Fusion Location

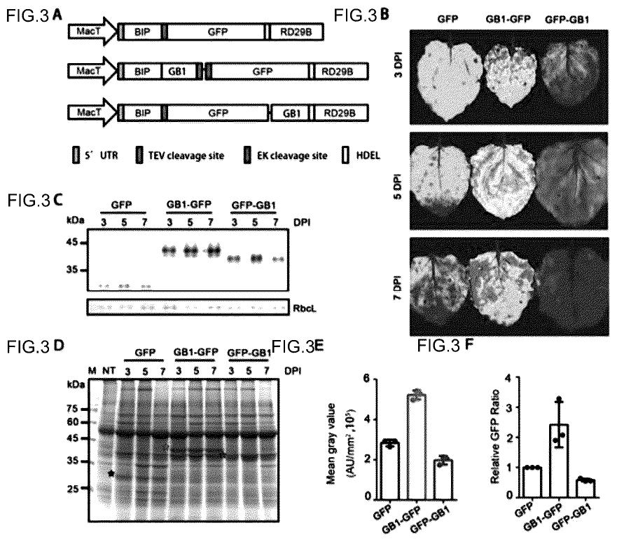

In order to verify the effect of upregulating protein expression depending on the fusion location of the GB1 domain, a BiP-GFP-GB1 construct was prepared in which the GB1 domain was fused to the C-terminus of GFP along with the endoplasmic reticulum retention signal sequence. Three Constructs of BiP-GFP, BiP-GB1-GFP and BiP-GFP-GB1 (see FIG. 3A) were transiently expressed in N. benthamiana, and the GFP expression level was confirmed by fluorescence imaging and Coomassie brilliant blue staining. The experimental method was performed in the same manner as in Example 2.

As a result, it was confirmed that when GB1 is fused to the C-terminus of GFP as shown in FIG. 3, it does not contribute at all to improving the amount of GFP expression compared to the case where GB1 is not fused to GFP. Thus, it was found that the location of the GB1 domain in the fusion protein had a significant effect on the protein expression level.

### Example 4. Verification of the Effect of Upregulating Target Protein Expression Using Human IL6 and HA of H9N2

In order to confirm whether the GB1 domain can upregulate the amount of protein expression for various target proteins, experiments were conducted using human interleukin 6 (hIL6) and H9N2 hemagglutinin (HA) as target proteins.

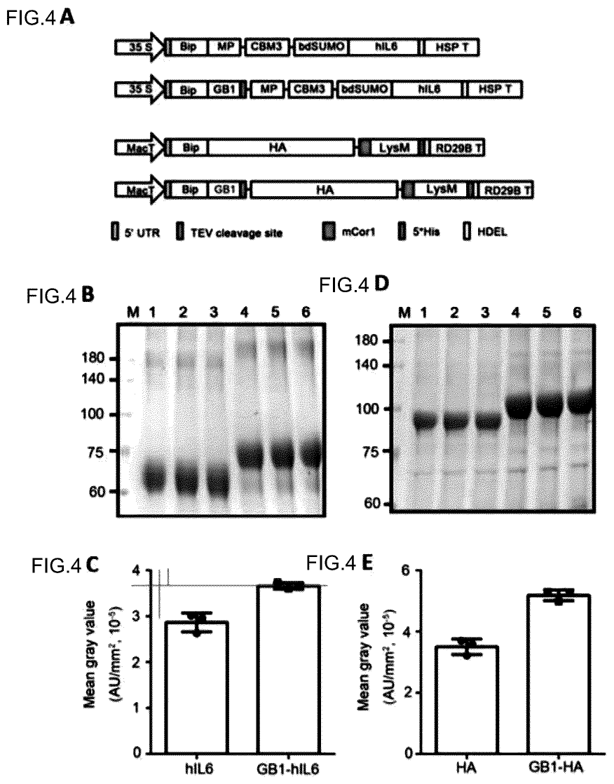

DNA constructs for plant expression of these two target proteins, BiP-MP-CBM3-SUMO-hIL6-HDEL (BiP-MCS-hIL6-HDEL), BiP-HA(H9N2)-mCor1-LysM-His-HDEL, BiP-GB1-MCS-hIL6-HDEL and BiP-GB1-HA(H9N2)-mCor1-LysM-His were prepared (see FIG. 4A), and were transiently expressed in leaf tissue of N. benthamiana through Agrobacterium-mediated infiltration.

The leaf extract of N. benthamiana was developed using SDS-PAGE, and then the expression of hIL6 and HA(H9N2) recombinant proteins was confirmed using anti-CBM3 (BioApp Co., Ltd.) and anti-His (Novus, AD1.1.10) antibodies, respectively. As a result, the recombinant protein fused with the GB1 domain showed much stronger signal intensity.

In order to quantify the expression level, hIL6 fused with GB1 domain and hIL6 without GB1 domain fusion were purified using microcrystalline cellulose (MCC) beads of CBM3 domain (Sigma-Aldrich). The protein bound to the MCC beads was separated from the MCC beads by boiling in SDS buffer, and then developed by SDS-PAGE. The gel was stained with Coomassie brilliant blue to quantify the intensity of the band.

As a result, the expression level of the hIL6 recombinant protein fused with the GB1 domain was 25% higher than that of the hIL6 recombinant protein to which the GB1 domain was not fused (FIGS. 4B and 4C).

Meanwhile, the HA (H9N2) recombinant protein was purified by using heat-inactivated Lactococcus (KCTC), and then the HA (H9N2) recombinant protein bound to the Lactococcus was separated by boiling in SDS buffer, and developed by SDS-PAGE. The gel was stained with Coomassie Brilliant Blue to identify HA(H9N2) recombinant protein, and the expression amount thereof was measured.

As a result, the GB1-fused HA(H9N2) recombinant protein showed a 50% higher expression level than HA(H9N2) without the GB1 domain fused (FIGS. 4D and 4E).

From these results, it was found that the target protein in which the GB1 domain is fused to the N-terminal has an improved expression level regardless of its type.

The sequences of human IL6 and H9N2 HA used in the present invention are as follows.

SEQ ID NO. 25: hIL6 amino acid sequence
MVPPGEDSKD VAAPHRQPLT SSERIDKQIR YILDGISALR KETCNKSNMC ESSKEALAEN 60
NLNLPKMAEK DGCFQSGFNE ETCLVKIITG LLEFEVYLEY LQNRFESSEE QARAVQMSTK 120
VLIQFLQKKA KNLDAITTPD PTTNASLLTK LQAQNQWLQD MTTHLILRSF KEFLQSSLRA 180
LRQM
SEQ ID NO. 26: hIL6 nucleotide sequence
atggtacccc caggagaaga ttccaaagat gtagccgccc cacacagaca gccactcacc 60
tcttcagaac gaattgacaa acaaattcgg tacatcctcg acggcatctc agccctgaga 120
aaggagacat gtaacaagag taacatgtgt gaaagcagca aagaggcact ggcagaaaac 180
aacctgaacc ttccaaagat ggctgaaaaa gatggatgct tccaatctgg attcaatgag 240
gagacttgcc tggtgaaaat catcactggt cttttggagt ttgaggtata cctagagtac 300
ctccagaaca gatttgagag tagtgaggaa caagccagag ctgtgcagat gagtacaaaa 360
gtcctgatcc agttcctgca gaaaaaggca aagaatctag atgcaataac cacccctgac 420
ccaaccacaa atgccagcct gctgacgaag ctgcaggcac agaaccagtg gctgcaggac 480
atgacaactc atctcattct gcgcagcttt aaggagttcc tgcagtccag cctgagggct 540
cttcggcaaa tg
SEQ ID NO. 27: H9N2 HA amino acid sequence
DKICIGYQST NSTETVDTLV ENNVPVTHTK ELLHTEHNGM LCATNLGHPL ILDTCTIEGL 60
VYGNPSCDLL LGGKEWSYIV ERSSAVNGMC YPGRVENLEE LRSFFSSARS YKRLLLFPDR 120
TWNVTFNGTS KACSGSFYRS MRWLTHKNNS YPIQDAQYTN DWGKNILFMW GIHHPPTDTE 180
QMNLYKKADT TTSITTEDIN RTFKPGIGPR PLVNGQQGRI DYYWSVLKPG QTLRIRSNGN 240
LIAPWYGHIL SGESHGRILK TDLNSGNCII QCQTEKGGLN TTLPFQNVSK YAFGNCPKYV 300
GVKSLKLAVG LRNVPATSGR GLFGAIAGFI EGGWPGLVAG WYGFQHSNDQ GVGIAADKES 360
TQEAVDKITS KVNNIIDKMN KQYEIIDHEF SEIEARLNMI NNKIDDQIQD IWAYNAELLV 420
LLENQKTLDD HDANVNNLYN KVKRALGSNA IEDGKGCFEL YHKCDDQCME TIRNGTYDRL 480
KYKEESKLER QKIEGVKLES EETYKI
SEQ ID NO. 28: H9N2 HA nucleotide sequence
gataaaattt gcattggcta ccagtcaaca aactccacag aaactgttga tacactagta 60
gaaaacaatg tccctgtgac acataccaaa gaattgctcc acacagagca caatggaatg 120
ttatgtgcaa caaacttggg acaccctctt atcctagaca cctgcaccat tgaagggttg 180
gtgtacggca atccttcctg tgatttgcta ctgggaggga aagagtggtc ttacattgtc 240
gaaagatcat cagctgttaa tgggatgtgc taccctggaa gggtagagaa tctggaagaa 300
ctcaggtcct ttttcagttc tgctcgctcc tacaaaagac tcctactttt tccagaccgt 360
acttggaatg tgactttcaa tgggacaagc aaagcatgct caggctcatt ctacagaagt 420
atgagatggc tgacacacaa gaacaattct taccctattc aagacgccca atataccaac 480
gactggggaa agaatattct cttcatgtgg ggcatacacc atccacctac tgatactgag 540
caaatgaatc tatacaaaaa agctgataca acaacaagta taacaacgga agatatcaat 600
cgaactttca aaccagggat agggccaagg cctcttgtca atggtcaaca aggaagaatt 660
gattattatt ggtcagtact aaagccaggc cagacattgc gaataagatc caatggaaat 720
ctaattgccc catggtatgg acacattctt tcaggagaaa gccatggaag aatcctgaag 780
accgatttga atagtggcaa ctgcataata caatgccaaa ctgagaaagg tggtttgaac 840
acgaccttgc cattccaaaa tgtcagcaaa tatgcatttg ggaactgtcc caaatatgtt 900
ggagtgaaga gtctcaaact ggcagttggt ctaaggaatg tgcctgctac atcaggtaga 960
gggcttttcg gtgccatagc tggattcata gaaggaggtt ggccaggact agttgcaggc 1020
tggtacgggt ttcagcactc aaatgatcaa ggggttggaa tagccgcaga caaagaatca 1080
actcaagaag cagttgataa aataacatcc aaagtaaata atataatcga caaaatgaac 1140
aagcagtatg aaatcattga tcatgagttc agtgagattg aagccagact caatatgatc 1200
aacaataaga ttgatgacca aatacaggac atctgggcgt acaatgcaga attactagta 1260
ctgcttgaaa accagaaaac actcgatgat catgatgcaa atgtgaacaa tctgtataat 1320
aaggtgaaga gagcattggg ttcaaatgca atagaggatg ggaagggatg cttcgagttg 1380
tatcacaaat gtgatgatca atgcatggaa acaattagaa acgggactta tgacaggcta 1440
aagtataaag aagaatcaaa actagaaagg cagaaaatag aaggggtaaa actggagtct 1500
gaagaaacat acaagatt

### Example 5. Confirmation of the Mechanism of GB1 Domain to Upregulate Expression of Target Protein

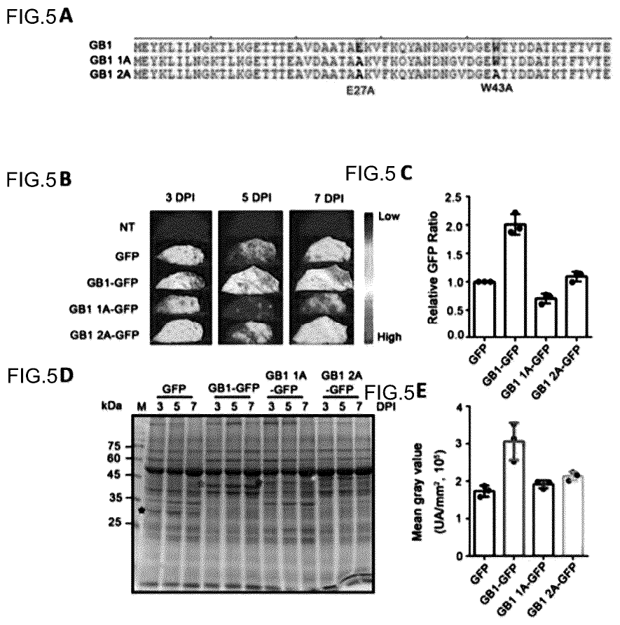

In order to identify the mechanism of the GB1 domain to upregulate expression of the target protein, E27 and W43, which are known to be important residues for GB1 binding to the Fc region of an antibody, were replaced with alanine using site-directed mutagenesis (see FIG. 5A), and it was examined what effect these variants have on enhancing the protein expression amount.

PCR was used for the site-directed mutation, and the primer sequences used were as follows.

TABLE 1

| | | | |
|---|---|---|---|
| E27A | overlap forward | gcggcgaccgcggcaaaa | SEQ ID |
| | primer | gttttcaaacagtatgc | NO. 29 |
| | overlap reverse | gcatactgtttgaaaact | SEQ ID |
| | primer | tttgccgcggtcgccg | NO. 30 |
| | end forward | gccttgcttcctattata | SEQ ID |
| | primer | tcttccc | NO. 31 |
| | end reverse | cccggatccttgaacctc | SEQ ID |
| | primer | ctgaacctccg | NO. 32 |
| W43A | overlap forward | cggtgtggatggtgaagc | SEQ ID |
| | primer | gacctacgatgatgc | NO. 33 |
| | overlap reverse | gcatcatcgtaggtcgct | SEQ ID |
| | primer | tcaccatccacaccg | NO. 34 |
| | end forward | gccttgcttcctattata | SEQ ID |
| | primer | tcttccc | NO. 35 |
| | end reverse | cccggatccttgaacctc | SEQ ID |
| | primer | ctgaacctccg | NO. 36 |

Each of the GB1 wild type and the variants thereof was fused to the GFP N-terminus, transiently expressed in N. benthamiana, and on days 3, 5, and 7 of expression, the degree of GFP fluorescence was confirmed and quantified. The total extract of N. benthamiana was developed by SDS/PAGE, confirmed by staining with Coomassie brilliant blue, and quantified.

As a result, as shown in FIG. 5, the effect of upregulating the expression of the target protein was not confirmed in each of the E27 and W43 variants of the GB1 domain (see FIG. 5), and thus, it was found that the residue in which GB1 binds to the Fc region of the antibody plays an important role in improving the expression of the target protein.

### Example 6. Expanded Verification of the Effect of GB1 Domain on Upregulating Target Protein Expression

The effect of the GB1 domain on upregulating expression of the target protein was sought to be confirmed in other plants. To this end, GFP and GB1-GFP constructs (see FIG. 1A) were introduced into protoplasts obtained from leaf cells of Arabidopsis by the PEG mediated transformation method (see Jin et al., 2001, Plant Cell, 13:1511-1526), and then total RNA was isolated from these protoplasts to perform qRT-PCR.

TABLE 2

| | | | |
|---|---|---|---|
| sGFP | forward | cagcagaac | SEQ ID NO. 37 |
| | primer | acccccatc | |
| | reverse | catgccgag | SEQ ID NO. 38 |
| | primer | agtgatccc | |
| Nicotiana | forward | atggaaacat | SEQ ID NO. 39 |
| benthamiana | primer | tgtgctcagt | |
| Actin | | g | |
| | reverse | ggtgctgaga | SEQ ID NO. 40 |
| | primer | gaagccaag | |

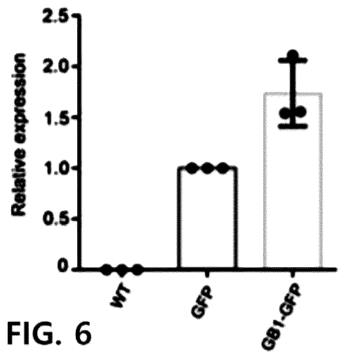

As a result, it was confirmed that when the GB1 domain was fused to the N-terminus as shown in FIG. 6, the expression level of the target protein increased by 50% or more even in Arabidopsis.

### Example 7. Confirmation of Stages in which the GB1 Domain Upregulates Target Protein Expression

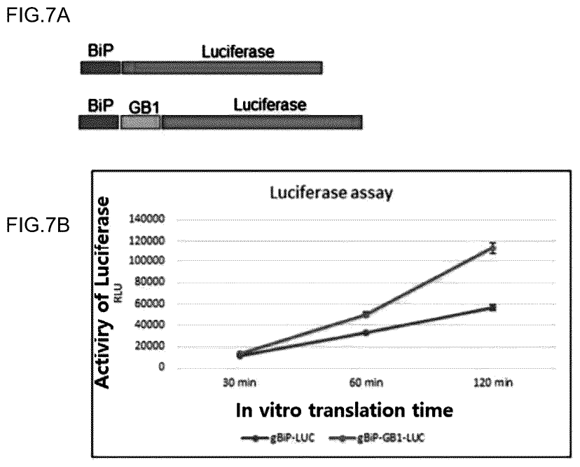

In order to confirm at which stage the GB1 domain upregulates the expression of the target protein, BiP:Luciferase and BiP:GB1:Luciferase constructs were introduced into pCS2++ vector to prepare constructs for in vitro translation (FIG. 7A). Linear form mRNA was created from the target gene by performing in vitro transcription according to the manufacturer's instructions using mMESSAGE mMACHINE SP6 (Invitrogen), and in vitro translation was performed according to the manufacturer's instructions using the Wheat Germ Extract kit (Promega). For an accurate experiment, 400 fmole of mRNA was added to each sample. The reaction was carried out at 25° C. with a total of 50 μL, and at 30, 60, and 120 minutes of reaction, 5 μL was aliquoted from each tube, diluted 24 times, and then quenched in liquid nitrogen. μL Luciferase assay was performed using the Dual-Luciferase Report Assay System (Promega) according to the manufacturer's instructions.

The amino acid and nucleotide sequences of luciferase used in the present invention are as follows:

SEQ ID NO. 41: Luciferase amino acid sequence
MEDAKNIKKG PAPFYPLEDG TAGEQLHKAM KRYALVPGTI AFTDAHIEVN ITYAEYFEMS 60
VRLAEAMKRY GLNTNHRIVV CSENSLQFFM PVLGALFIGV AVAPANDIYN ERELLNSMNI 120
SQPTVVFVSK KGLQKILNVQ KKLPIIQKII IMDSKTDYQG FQSMYTFVTS HLPPGFNEYD 180
FVPESFDRDK TIALIMNSSG STGLPKGVAL PHRTACVRFS HARDPIFGNQ IIPDTAILSV 240
VPFHHGFGMF TTLGYLICGF RVVLMYRFEE ELFLRSLQDY KIQSALLVPT LFSFFAKSTL 300
IDKYDLSNLH EIASGGAPLS KEVGEAVAKR FHLPGIRQGY GLTETTSAIL ITPEGDDKPG 360
AVGKVVPFFE AKVVDLDTGK TLGVNQRGEL CVRGPMIMSG YVNNPEATNA LIDKDGWLHS 420
GDIAYWDEDE HFFIVDRLKS LIKYKGYQVA PAELESILLQ HPNIFDAGVA GLPDDDAGEL 480
PAAVVVLEHG KTMTEKEIVD YVASQVTTAK KLRGGVVFVD EVPKGLTGKL DARKIREILI 540
KAKKGGKSKL
SEQ ID NO. 42: Luciferase nucleotide sequence
caaatggaag acgccaaaaa cataaagaaa ggcccggcgc cattctatcc tctagaggat 60
ggaaccgctg gagagcaact gcataaggct atgaagagat acgccctggt tcctggaaca 120
attgctttta cagatgcaca tatcgaggtg aacatcacgt acgcggaata cttcgaaatg 180
tccgttcggt tggcagaagc tatgaaacga tatgggctga atacaaatca cagaatcgtc 240
gtatgcagtg aaaactctct tcaattcttt atgccggtgt tgggcgcgtt atttatcgga 300
gttgcagttg cgcccgcgaa cgacatttat aatgaacgtg aattgctcaa cagtatgaac 360
atttcgcagc ctaccgtagt gtttgtttcc aaaaaggggt tgcaaaaaat tttgaacgtg 420
caaaaaaaat taccaataat ccagaaaatt attatcatgg attctaaaac ggattaccag 480
ggatttcagt cgatgtacac gttcgtcaca tctcatctac ctcccggttt taatgaatac 540
gattttgtac cagagtcctt tgatcgtgac aaaacaattg caataatgaa ttcctctgga 600
tctactgggt tacctaaggg tgtggccctt ccgcatagaa ctgcctgcgt cagattctcg 660
catgccagag atcctatttt tggcaatcaa atcattccgg atactgcgat tttaagtgtt 720
gttccattcc atcacggttt tggaatgttt actacactcg gatatttgat atgtggattt 780
cgagtcgtct taatgtatag atttgaagaa gagctgtttt tacgatccct tcaggattac 840
aaaattcaaa gtgcgttgct agtaccaacc ctattttcat tcttcgccaa aagcactctg 900
attgacaaat acgatttatc taatttacac gaaattgctt ctgggggcgc acctctttcg 960
aaagaagtcg gggaagcggt tgcaaaacgc ttccatcttc cagggatacg acaaggatat 1020
gggctcactg agactacatc agctattctg attacacccg agggggatga taaaccgggc 1080
gcggtcggta aagttgttcc attttttgaa gcgaaggttg tggatctgga taccgggaaa 1140
acgctgggcg ttaatcagag aggcgaatta tgtgtcagag gacctatgat tatgtccggt 1200
tatgtaaaca atccggaagc gaccaacgcc ttgattgaca aggatggatg gctacattct 1260
ggagacatag cttactggga cgaagacgaa cacttcttca tagttgaccg cttgaagtct 1320
ttaattaaat acaaaggata tcaggtggcc cccgctgaat tggaatcgat attgttacaa 1380
caccccaaca tcttcgacgc gggcgtggca ggtcttcccg acgatgacgc cggtgaactt 1440
cccgccgccg ttgttgtttt ggagcacgga aagacgatga cggaaaaaga gatcgtggat 1500
tacgtcgcca gtcaagtaac aaccgcgaaa aagttgcgcg gaggagttgt gtttgtggac 1560
gaagtaccga aaggtcttac cggaaaactc gacgcaagaa aaatcagaga gatcctcata 1620
aaggccaaga agggcggaaa gtccaaattg taa

As a result of the experiment, it was found that the BiP:GB1:Luciferase showed twice the activity of the BiP:luciferase at the reaction time of 120 minutes (FIG. 7B), and the GB1 domain was able to improve the expression amount of the target protein at both the transcription stage (FIG. 6) and the translation stage (FIG. 7).

Although the specific contents of the present invention have been described in detail above, it will be clear to those skilled in the art that these specific descriptions are merely preferred embodiments, and do not limit the scope of the present invention. Accordingly, it can be said that the substantial scope of the present invention is defined by the appended claims and their equivalents.

1: A fusion protein comprising: a target protein; and a GB1 domain bound to the N-terminus of the target protein.

2: The fusion protein according to claim 1, wherein the GB1 domain is represented by the amino acid sequence of SEQ ID NO. 1.

3: The fusion protein according to claim 1, further comprising a cleavage site between the target protein and the GB1 domain.

4: The fusion protein according to claim 1, wherein the fusion protein further comprises an intracellular organelle targeting sequence.

5: A DNA construct comprising a nucleotide sequence encoding the fusion protein of claim 1.

6: The DNA construct according to claim 5, wherein the DNA construct further comprises a 5′ UTR sequence at the 5′-terminal site of the nucleotide sequence encoding the fusion protein.

7: The DNA construct according to claim 5, wherein the GB1 domain is fused to the N-terminus of the target protein to increase the expression amount of the target protein in plants.

8: A plant cell into which the DNA construct of claim 5 or a recombinant vector comprising the DNA construct of claim 5 is introduced.

9: The plant cell according to claim 8, wherein the plant cell is derived from a plant selected from a group consisting of Arabidopsis thaliana, soybean, tobacco, eggplant, red pepper, potato, tomato, cabbage, radish, cabbage, lettuce, peach, pear, strawberry, watermelon, melon, cucumber, carrot, celery, rice, barley, wheat, rye, corn, sugarcane, oats, and onions.

10: A method for producing a target protein in a plant cell, the method comprising the steps of:
(a) culturing the plant cell of claim 8; and
(b) recovering the target protein by crushing the cultured plant cell.

11: The method according to claim 10, wherein when the DNA construct further comprises a cleavage site between the target protein and the GB1 domain, the target protein and the GB1 domain is cleaved to recover the target protein from which the GB1 domain has been removed.

12: A transgenic plant into which the DNA construct of claim 5 or a recombinant vector comprising the DNA construct of claim 5 is introduced.

13: The transgenic plant according to claim 12, wherein the transgenic plant is selected from a group consisting of Arabidopsis thaliana, soybean, tobacco, eggplant, red pepper, potato, tomato, Chinese cabbage, radish, cabbage, lettuce, peach, pear, strawberry, watermelon, melon, cucumber, carrot, celery, rice, barley, wheat, rye, corn, sugarcane, oats, and onions.

14: A method for producing a target protein in a transgenic plant, the method comprising the steps of:
(a) growing the transgenic plant of claim 12; and
(b) recovering the target protein by crushing the tissue isolated from the plant.

15: The method according to claim 14, wherein when the DNA construct further comprises a cleavage site between the target protein and the GB1 domain, the target protein and the GB1 domain is cleaved to recover the target protein from which the GB1 domain has been removed.
